(19) United States
(12) Patent Application Publication
(10) Pub. No.: US 2004/0120995 A1
(43) Pub. Date: Jun. 24, 2004

(54) Transdermal delivery of pergolide

(76) Inventors: Debra A Martin, Sunrise, FL (US); Jesus Miranda, Miami, FL (US); Adolfo Leonel Vanegas, Miami, FL (US)

Correspondence Address: H Eric Fischer, Synnestvedt & Lechner, 2600 Aramark Tower, 1101 Market Street, Philadelphia, PA, 19107-2950, US

(21) Appl. No.: 10/473,511

(22) PCT Filed: Apr. 1, 2002
(86) PCT No.: PCT/US02/10057

(51) Int. Cl. A61K 9/70

(52) U.S. Cl. 424/449

(57) ABSTRACT

A transdermal delivery device comprising a multi-phase matrix which includes a solid liquid-retaining member and associated therewith pergolide in liquid form, for example, a transdermal delivery device comprising a barrier layer affixed to one face of the matrix and having an exposed face, the exposed face having affixed thereto a mounting layer, and the other face of the matrix having affixed thereto a removable layer, an example of the liquid-retaining member being an absorbent material, for example, a natural or synthetic fiber gauze, a non-woven medical basorbent, a natural sponge, and a synthetic sponge.

This application claims the benefit of the filing date of U.S. Provisional Application No. 60/280,533, filed Mar. 30, 2001.

### FIELD OF THE INVENTION

This invention relates to the administration of pergolide to a patient. More particularly, this invention relates to the transdermal delivery of pergolide.

Pergolide is a drug that is used to treat various health conditions that affect individuals. For example, pergolide has been used in the treatment of the symptoms of Parkinson's disease and in the reduction of plasma concentrations of prolactin in conjunction with the treatment of hyperprolactinemia. In the treatment of Parkinson's disease, the typical course of medication is a gradual increase in the oral dose over 14 days, with a concomitant gradual increase in blood serum levels. Blood serum levels ranging between a maximum of about 500 picograms/ml (maximum serum level 2-3 hours following a 2.5 mg oral dose) to about 60 picograms/ml (serum level 8 hours following a 0.5 mg oral dose) have been considered within a therapeutic range from oral dosing studies of pergolide mesylate in the treatment of Parkinson's disease. Typically, oral dosing is 3 mg/24 hours, divided into three doses, with serum concentrations peaking at approximately 200 pg/ml.

The oral administration of pergolide offers the advantage of being simple to administer. However, with oral administration, blood serum levels fluctuate between dosages and the drug must pass through the liver before systemic distribution in the blood stream, requiring a dosage level high enough to account for metabolic losses in the hepatic system. Accordingly, there are disadvantages associated with the to administration of oral doses of pergolide.

It is known also to administer pergolide by transdermal delivery, such means of delivery having advantages relative to oral delivery. The present invention relates to the transdermal delivery of pergolide.

Reported Developments

European published patent application EP 0913128A1 discloses a transdermal device for delivering a variety of medicaments, for example, pergolide. The device utilizes a layer of polymeric adhesive in which the medicament is dispersed homogeneously. The medicament-containing adhesive layer is prepared by dissolving the constituents in a solvent(s). The resulting solution solidifies to form the medicament-containing layer upon evaporation of the solvent. The device includes also a backing layer on one side of the adhesive layer to protect the adhesive layer during use and storage and a strippable release layer on the other side of the adhesive layer to protect the otherwise exposed side of the adhesive layer during storage. The strippable layer is removed before application of the adhesive layer of the device to the body membrane.

Transdermal delivery of pergolide is disclosed also in U.S. Pat. No. 6,001,390 to Yum. The '390 patent discloses the delivery of pergolide mesylate in vitro across samples of human skin. In one embodiment of the delivery devices disclosed in the Yum patent, a liquid pergolide-containing composition is held within the device in a space from which the pergolide is delivered to the body membrane by diffusion through a micro-porous membrane in contact therewith. The device includes a strippable layer which covers the face of the micro-porous membrane that is placed in contact with the body membrane and which is removed at the time of use. In another embodiment disclosed in the Yum patent, the pergolide is contained in a solid polymeric film which is prepared by forming a solution of the pergolide and polymeric carrier and evaporating the solvent to form the polymeric film.

One of the shortcomings of the present means for delivering pergolide transdermally is that relatively large amounts of pergolide must be used in the delivery device in order to achieve desired therapeutic pergolide blood serum levels. The present invention relates to improved means for the transdermal delivery of pergolide to a patient.

### SUMMARY OF THE INVENTION

In accordance with the present invention, there is provided a transdermal delivery device comprising a multi-phase matrix which includes a solid liquid-retaining member and associated therewith pergolide in liquid form. In a preferred embodiment, the liquid-retaining member is, for example, a non-woven medical absorbent and the pergolide is dissolved in an aqueous based solvent.

Another aspect of the present invention is the provision of a method for the transdermal delivery of pergolide comprising delivering to a body membrane pergolide in liquid form and from a liquid pergolide-carrying member which is in direct contact with the body membrane. In this embodiment of the invention, the pergolide is delivered from a supply source directly to the body membrane unimpeded by a material interposed between the source and the surface of the body membrane.

The present invention provides the means to efficiently deliver relatively high amounts of pergolide transdermally and to achieve desired blood serum levels of the pergolide. And this can be accomplished without irritating the membrane of the patient.

### BRIEF DESCRIPTION OF THE DRAWINGS

FIGS. 1 to 4 are graphical representation of in vitro pergolide delivery flux from a transdermal delivery device of the present invention.

FIG. 5 is a graphical representation of in vivo pergolide blood serum levels attained by a transdermal delivery device of the present invention.

FIG. 6 is a graphical representation of in vitro pergolide delivery flux involving the use of a comparative composition comprising a solid solution of pergolide.

FIGS. 7 to 9 are graphical representations of in vitro pergolide delivery flux from direct application to the skin of a liquid pergolide composition.

### DETAILED DESCRIPTION OF THE INVENTION

The present invention relates to the transdermal delivery of liquid pergolide from a transdermal delivery device held in contact with a membrane of an animal to which pergolide is to be delivered. The delivery device of the present invention comprises a multi-phase matrix which includes a solid material for holding the liquid pergolide. The device may include other elements which relate to the use or functioning of the device. Examples of such elements are a layer to secure the device in place during use, a barrier layer on that surface of the matrix which is not in contact with the body membrane to prevent the liquid pergolide from exiting that surface, and a release layer which shields the other surface of the matrix from the ambient when the device is not in use.

The multi-phase matrix of the delivery device has at least two elements, pergolide in liquid form and a solid material which has an affinity for the liquid and so functions as a liquid-retaining member. The affinity between the composition comprising the liquid pergolide and the solid liquid-retaining member may be due, for example, to adsorption such as physi- or chemi-sorption or to absorption within void spaces in the material.

Typically, the liquid pergolide is contained in a composition which can include other constituents, as described below. The composition is held by the liquid-retaining member with sufficiently strong force to withstand pressure that the device may be subjected to in the ordinary course of use and handling without expelling the composition therefrom.

The liquid-retaining member of the delivery device of the present invention can be made of any suitable material. Speaking generally, the material should exhibit an affinity for some or all of the components of the composition which comprises the liquid pergolide. The material should not irreversibly react or interact with any of the components of the composition. For example, it should resist being degraded by the composition. Conversely, the material should not affect adversely any components of the composition. In this respect, materials which are inert toward the components of the composition and do not dissolve therein are suitable materials for use in fabricating the liquid-retaining member. Examples of suitable materials are reticulated materials represented by the natural and synthetic fibers in the form of woven gauze, relatively long staple absorbent masses, non-woven fiber web, and a non-woven web which is surface-treated with a porous polymer. A preferred material is a non-woven material comprising rayon web covered with porous polyethylene, for example, 1603 non-woven medical absorbent available from 3M.

Such materials are capable of retaining the composition comprising the liquid pergolide in cavities, pores or channels that comprise the materials. The liquid-retaining member can also comprise a material which adsorbs the liquid pergolide. For example, a surface of a component of the liquid composition can be adsorbed to a surface of the material comprising the liquid-retaining member and, by cohesive interaction with un-adsorbed components of the liquid composition, the bulk of the composition is retained by the member.

The solid liquid-retaining member of the delivery device of the present invention has associated therewith pergolide in liquid form. The term “pergolide” is used herein to mean any pharmaceutically acceptable species of ergoline having pharmaceutical properties like those of the free base 8 β-[(methylthio)methyl]-6-propyl ergoline. It is recognized that there are many species of pergolide which are suitable for the treatment of abnormal bodily conditions, for example, treating the symptoms of Parkinson's disease and hyperprolactinemia. In addition to the aforementioned free base, acid salts of the free base and compounds which have structural variations of the ergoline ring are known to be pharmaceutically active. Examples of pharmaceutically acceptable salts of pergolide and compounds with structural variations of the ergoline ring that exhibit pharmacological properties and that may be used in the practice of the present invention are disclosed in U.S. Pat. No. 4,166,182 to Kornfeld et. al. A mixture of two or more species of pergolide may be used in the practice of the present invention.

Preferred forms of pergolide for use in the practice of the present invention are pergolide mesylate and pergolide free-base, both of which are a solid at room temperature and are sufficiently soluble in water or nonaqueous solvents to provide a solution which contains pharmaceutically effective amounts of the dissolved pergolide species.

Many of the species of pergolide suitable for use in the present invention are solids at ambient temperatures. Accordingly, the matrix of the delivery device will typically contain a composition which comprises a “pergolide” solvent in addition to the pergolide. It is likely that a solvent will be used also with those pharmaceutically acceptable species of pergolide that are liquids under ambient conditions to function as a diluent or carrier of the liquid pergolide

Any suitable liquid solvent (inorganic or organic) which is capable of dissolving the pergolide in an amount which is considered sufficient for including in the matrix of the delivery device can be used. Water, alcohols, for example, ethanol, dimethyl sulfoxide (DMSO), and glycols, for example, polyethylene glycol and polypropylene glycol are examples of solvents that can be used.

It is known that skin and mucous membranes are irritated by direct application thereto of a relatively high amount of pergolide. The “pergolide” solvent serves also as a diluent and functions to reduce undesirable irritation. The solvent can act also to improve the permeability of the skin or mucous membrane to the pergolide. In addition, the solvent can function as a diffusion media which helps to conduct the pergolide to the body membrane through which it enters the body.

The pergolide-containing composition can comprise a single phase composition, for example, a liquid solution of pergolide, or it can comprise a multi-phase composition, for example, an emulsion, a gel, or a dispersion which includes the pergolide in liquid form. An emulsion can comprise liquid droplets of a solution of pergolide dispersed in a continuous liquid phase A gel can comprise a phase of continuous solution thickened by an appropriate gelling agent which comprises a dispersed phase. A dispersion can comprise a liquid solution of dissolved pergolide having dispensed therein solid particles of pergolide, for example, nanoparticles of pergolide.

In preferred form, the composition comprises a solution of pergolide having a viscosity at room temperature such that the solution flows readily, for example, from a pipette which is used to deliver the solution to the liquid-returning member of the matrix. An aqueous-based solution which includes a hydrocarbon-based solvent is particularly preferred.

The pergolide-containing composition can include one or more additives which function to impart desired properties to the composition. For example, the composition can include a co-solvent to improve the solubility of the pergolide in the principal solvent. The use of an alcohol as a co-solvent to improve the solubility of pergolide in water is exemplary. The composition can include an enhancer which functions to enhance the ability of the pergolide to be delivered transdermally. Examples of enhancers are alcohols, glycols, fatty acids, and fatty acid esters. Another example of an additive is a thickening agent, for example, hydroxymethyl cellulose, which functions to impart to the composition a desired viscosity.

Other exemplary additives are, for example, stabilizers, preservatives, and antioxidants. A stabilizer is an art-recognized compound defined in the “Handbook of Pharmaceutical Additives,” Ash, Michael and Irene, Gower 1995, to be a pharmaceutical additive that thickens, prevents separation, retards oxidation by increasing viscosity, and gives a smoother product. An antioxidant is also an art-recognized compound and is defined by the Handbook of Pharmaceutical Additives to be a substance that retards oxidation, deterioration, rancidity, and gum formation in organic substances. A preservative is also an art-recognized compound defined by the Handbook of Pharmaceutical Additives to be a substance, either natural or synthetic, that protects a pharmaceutical composition against spoilage, discoloration, or decay and is used to retard or prevent microbial or chemical spoilage. The “Handbook of Pharmaceutical Excipients,” Kibbe H. Arthur, 3^{rd} ed. American Pharmaceutical Association 2000 lists many compounds which are recognized: as stabilizers, for example, L-Methionine; as antioxidants, for example, citric acid, ascorbic acid, butylated hydroxy anisole (BAH), and butylated hydroxy toluene (BHT); and as preservatives, for example, benzyl alcohol, ethyl alcohol, and citric acid. Compositions of the present development can include also one more of such compounds.

Some additives may improve more than one property of the composition. For example, DMSO may enhance the solubility of the pergolide and its ability to be delivered transdermally.

Additives of the type referred to herein, as well as other additives for use in pergolide compositions, are known. Accordingly, it should be understood that compounds other than those referred to above can be used in the pergolide-containing composition. In preferred form, the additives are present in dissolved form in the composition.

The amount of pergolide comprising the composition should be an amount sufficient to deliver transdermally a pharmaceutically effective amount of pergolide to the body. Such amount will vary depending on numerous factors, for example, the species of pergolide used, the condition to be treated, the nature of the material comprising the liquid-retaining member of the matrix, and the area of the matrix surface which is in contact with the body membrane. It is believed that a composition comprising about 0.1 to about 10 wt. % of pergolide will be effective for most applications. However, it should be understood that there may be applications where the composition comprises a lower or higher proportion of pergolide.

Similarly, the amount of any particular additive comprising the composition will depend on numerous factors. For most applications, it is believed that the additive will typically comprise about 0.01 to about 50 wt. % of the composition. Lower or higher amounts can be used, depending on the particular additive and the involved application. For example, an additive such as a stabilizer, preservative, or an antioxidant can comprise about 0.4 to about 2 wt. %of the composition.

A preferred composition comprising about 90 to about 96 wt. % water, about 0.4 to about 2 wt. % β-cyclodextrine, about 3 wt. % to about 6 wt. % hydroxy propyl cellulose, and about 0.1 wt. % to about 1 wt. % pergolide mesylate is particularly useful both as a form of liquid pergolide and as a base composition to which stabilizers, antioxidants, and preservatives can be added to yield a liquid pergolide composition. Addition of up to about 0.4 wt. % of an antioxidant, for example, about 0.05 to 0.4 wt. % ascorbic acid and/or citric acid, and/or up to about 4 wt % of a preservative, for example, about 0.05 to about 4 wt. % benzyl alcohol and/or lactic acid, has been found to yield a particularly stable composition which is particularly preferred.

Another preferred composition includes up to about 5 wt. % ethyl alcohol (for example, about 1 to about 5 wt. %) which can be added to the base composition as a preservative and co-solvent and to which additional antioxidants, stabilizers, and preservatives can be added to yield a composition which is particularly stable.

The matrix of the present invention may be prepared by selecting a liquid-retaining material which has a suitable affinity for the pergolide-containing composition, forming it or cutting it into a desired shape which has surface area of desired magnitude and applying, to the material the desired amount of composition. Alternatively, the composition can be applied to a bulk of the liquid-retaining material which is then shaped or cut to the desired size. Any suitable shape of retaining material can be used. Typically the material is in the form of a disk or pad having two faces and an edge.

The transdermal delivery device of the present invention can include other elements of the type that are normally present in transdermal delivery devices including, for example, a barrier layer which covers. one of the faces of the matrix, a layer which holds the device to the body surface to be treated, and a removable layer that protects the other face of the matrix and which can be removed when the device is ready for use.

In preferred form, the liquid-retaining material of the matrix is in direct contact with the body membrane during use. However, there may be applications where it is useful to include in the device a permeable layer, for example, a microporous membrane, through which the pergolide is capable of flowing on its way to the body surface and which covers that face of matrix membrane which faces toward the body membrane. In this type of embodiment, the face of the permeable layer not in contact with the face of the matrix which can be covered with the removable layer.

In accordance with the present invention, transdermal delivery devices were fabricated and pergolide delivery flux was determined from their use in separate in vitro and in vivo measurements. In vitro measurements of pergolide delivery flux in all cases were carried out using a diffusion cell to which samples of human skin were fixed such that the stratum corneum was accessible for application of the delivery device or of a composition comprising pergolide in liquid form.

In a typical determination, a skin sample was fastened onto a diffusion cell filled with saline solution as a receiver liquid. After application of either an aliquot of a liquid pergolide-containing composition or a transdermal delivery device of the present invention to the stratum corneum of the affixed sample, the cell was placed in an environment in which an even temperature was maintained throughout the evaluations.

In a typical determination, the diffusion cell was equilibrated for, a predetermined time then a sample of the receiver liquid was withdrawn to establish a baseline. Subsequent samples of receiver liquid were removed at predetermined time intervals.

### EXAMPLES

The first group of examples are illustrative of transdermal delivery devices of the present invention. They include a multi-phase matrix comprising a liquid-retaining material in the form of disks of the various materials listed below in Table 1 (Example Nos. 1-8) and a phase of pergolide in liquid form covering an area of 1 cm² .

The liquid pergolide used in the devices of Example Nos. 1-5 of Table 1 was a viscous liquid composition comprising 0.48 wt. % pergolide mesylate, 0.8 wt. % β-cyclodextrin, 3.0 wt. % hydroxypropyl cellulose, and 95.7 wt. % water; this is a particularly preferred composition. The device of Example No. 6 included a liquid composition comprising 0.5 wt. % pergolide mesylate, 3.0 wt. % hydroxypropyl cellulose, and 96.5 wt. % water. The device of Example No. 7 included a liquid composition comprising 0.55 wt. % pergolide mesylate, 6.0 wt. % hydroxypropyl cellulose, and 93.45 wt. % water, and that of Example No. 8 included a composition comprising 0.05 wt. % pergolide mesylate and 99.95 wt. % water.

Each of the compositions used in the transdermal devices of the examples was made by dissolving solid particles of pergolide mesylate in deionized water and adding β cyclodextrin (if used) and hydroxypropyl cellulose (if used) to yield the wt. % composition listed. The compositions were of sufficiently low viscosity to be dispensed onto the liquid-retaining material from a conventional pipette.

All of the examplary transdermal delivery devices described herein were fabricated by forming the selected liquid-retaining material into a desired shape and area (for example, a pad or a disk) and then laminating one face of the shaped material to a section of 9732 polyester film (3M) which functioned as a barrier layer. The film was sized so that the barrier layer of the resulting laminate extended beyond the edges of the retaining material. The laminate was affixed to the adhesive face of 9772 PVC foam tape (3M) such that the barrier layer was interposed between the tape and the material. The PVC foam tape was sized to extend beyond the edges of the barrier layer so that the tape functioned as an adhesive layer to hold the laminate in contact with a membrane to which the device was applied.

The pergolide composition was applied to the exposed surface of the retaining material over a controlled area of surface. The area to which the pergolide composition was applied was selected to give an area of desired size over which transdermal delivery could occur (active area). The pergolide composition was applied in an amount of 70 μl/cm² of the selected area. For the devices of Example Nos. 1-5 of Table 1, this yielded devices having approximately 0.346 mg pergolide mesylate/cm² of liquid-retaining material. The devices of Example Nos. 6 and 7 contained 0.89 mg pergolide mesylate/cm² of liquid-retaining materials and that of Example No. 8 contained 0.07 mg pergolide mesylate /cm² of liquid-retaining material . The area stated for the liquid-retaining material is the area of the pad to which the liquid composition was applied measured as superficial area.

After application of the liquid composition to the liquid-retaining material, its exposed surface matrix and the exposed adhesive face of the PVC foam tape were covered with a release liner made from 164Z Polyester-daubert release liner (Sano Corp.). Prior to use, the release liner was stripped from the transdermal device and the exposed surface of the liquid-retaining material and foam tape were applied to a section of human skin mounted on a diffusion cell.

The devices were subjected to in vitro pergolide delivery flux determinations according to the method described herein above. The results are summarized next to each entry in Table 1, which also references the Figures in which additional data from the in vitro determinations is presented.

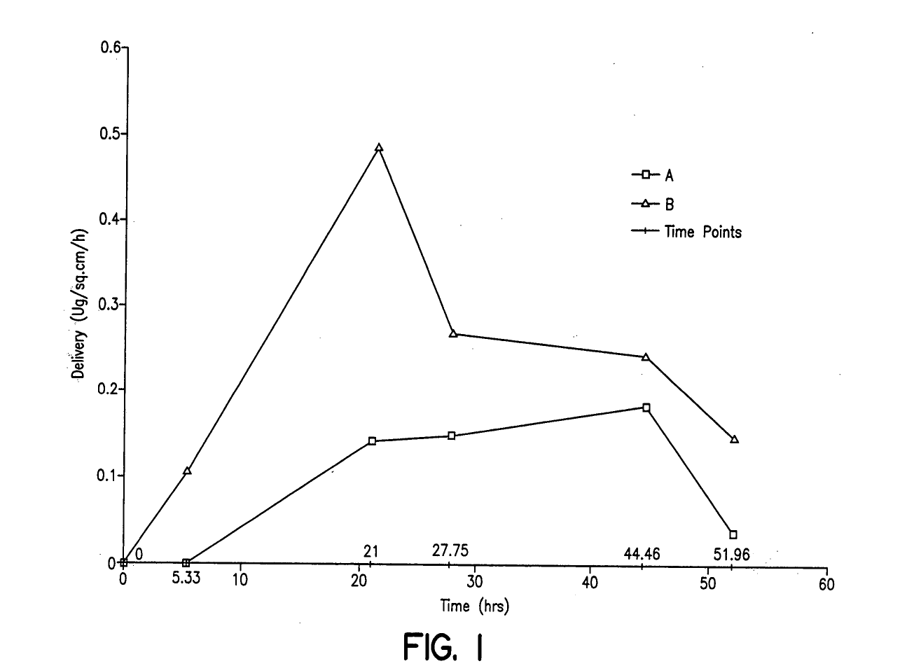

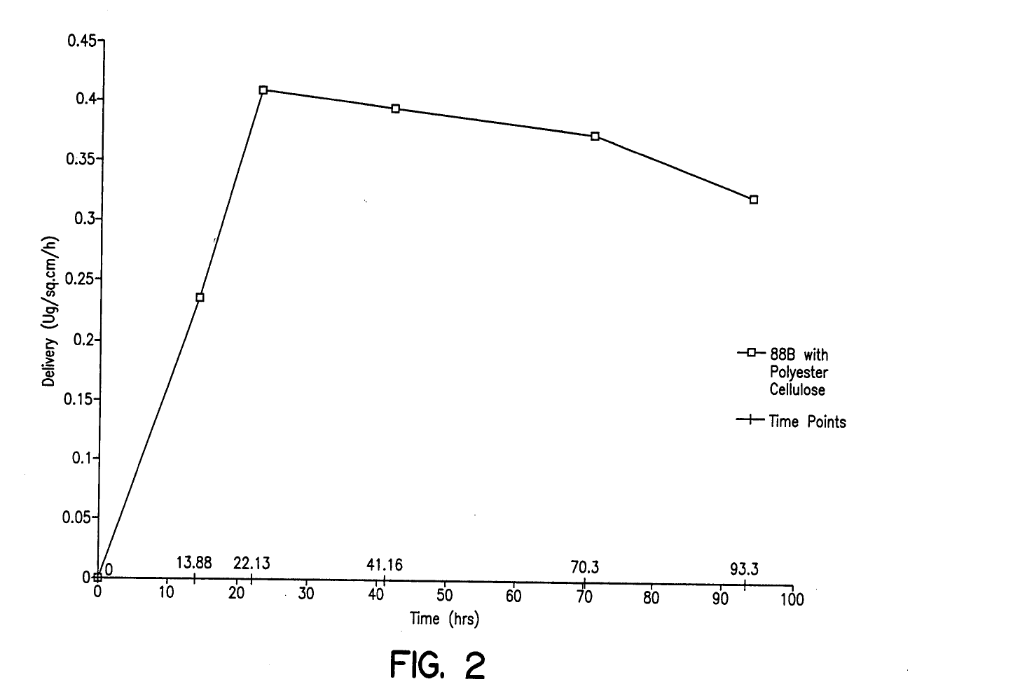

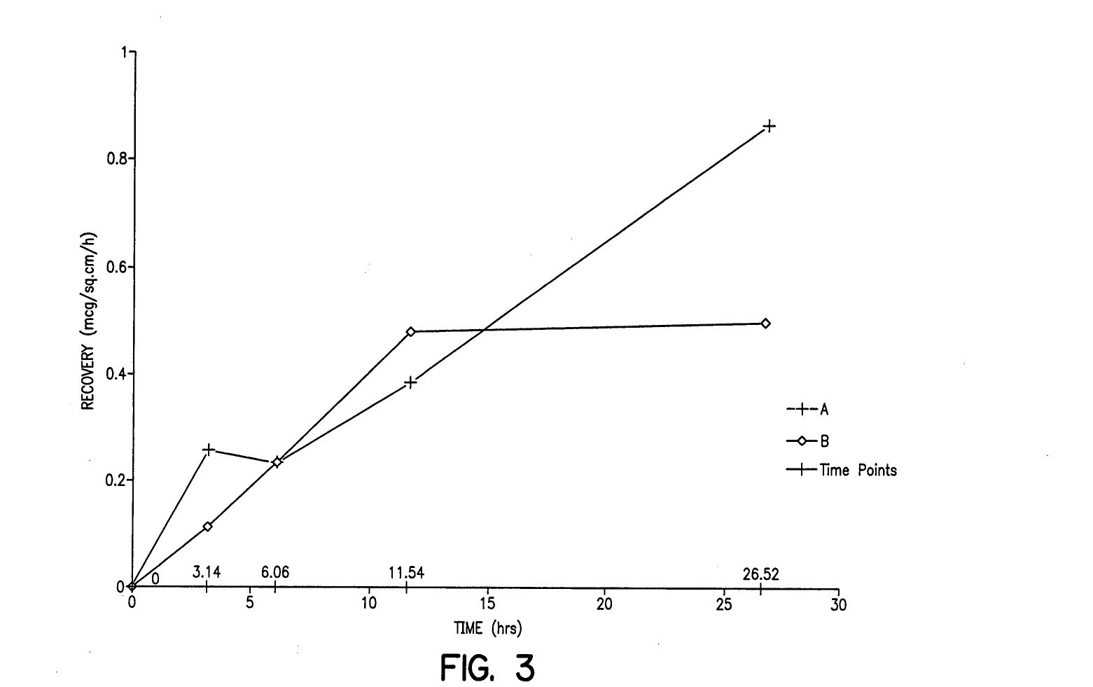

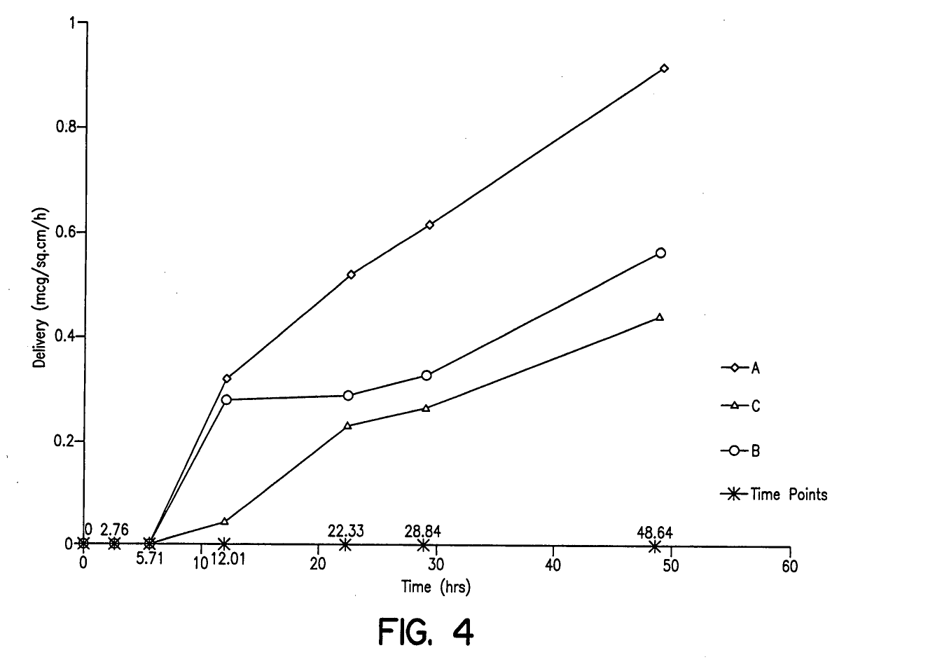

TABLE 1

| | | PERGOLIDE DELIVERY FLUX | | |
|---|---|---|---|---|
| | | [micrograms cm⁻² hr⁻¹] | | |
| | Liquid-retaining material | INITIAL | PEAK | STEADY STATE |
| Ex. No. | FIG. # | flux/time* | flux/time* | flux/time* |
| 1 | 3 M 1603 non-woven | 0.24/14 hrs | 0.41/22 hrs | 0.34-0.40/22-70 hrs. |
| | No FIG. | | | |
| 2 | 3 M 1603 non-woven | 0.00/5 hrs | 0.19/45 hrs | 0.15-0.19/21-45 hrs. |
| | FIG. 1 - A | | | steady rise |
| 3 | Veratec 2008080 | 0.11/5 hrs | 049/21 hrs | 0.28-0.25/28-45 hrs. |
| | FIG. 1 - B | | | steady decrease |
| 4 | Veratec 9408807 | 0.22/14 hrs | 0.41/22 hrs | 0.42-0.37/22-70 hrs. |
| | | | | steady decrease |
| 5 | 3 M 1603 non-woven | Membrane a | 0.5/11.5 hrs | 0.5/11.5-26.5 hrs |
| | | 0.1/3 hrs. | | |
| | | FIG. 3 - A | | |
| | | Membrane b | 0.85/26.5 hrs | no steady state |
| | | 0.25/3 hrs | | 0.25-0.85/steady |
| | | FIG. 3 - B | | increase 11.5-26.5 hrs. |
| 6 | 3 M 1603 non-woven | 0.0/at app. | 0.90/49 hours (end | 0.35-0.90/steady rise |
| | FIG. 4 - A | | of test - no peak) | 12-49 hours, no steady |
| | | | | state |
| 7 | 3 M 1603 non-woven | 0.0/at app. | 0.55/49 hours (end | 0.30-0.55/steady rise |
| | FIG. 4 - B | | of test - no peak) | 12-49 hours, no steady |
| | | | | state |
| 8 | 3 M 1603 non-woven | 0.0/at app. | 0.45/49 hours (end | 0.05-0.45/steady rise |
| | FIG. 4 - C | | of test - no peak) | 12-49 hours, no steady |
| | | | | state |

*Time from initial contact of pergolide source to membrane

The data in Table 1 above shows that pergolide can be delivered from a transdermal delivery device of the present invention at a delivery flux commensurate with establishing therapeutic blood serum levels of pergolide.

The next group of examples illustrate the use on human subjects of delivery devices similar to those described above. The liquid-retaining material of the devices comprised pads of 1603 non-woven medical absorbent (3M) having surface areas sufficient to have a liquid form of pergolide applied to a 10 or 30 cm² area. The devices included the same composition as that used in the devices of Example Nos. 1-6 of Table 1 (0.48 wt. % pergolide mesylate, 0.8 wt. % β-cyclodextrin, 3.0 wt. % hydroxypropyl cellulose, and 95.7 wt. % water). Twelve devices having a 10 cm² active area and 4 devices having a 30 cm² active area were prepared. One device having a 10 cm² active area was applied to four subjects. Four other subjects received two devices each having a 10 cm² active area, and four other subjects each received a device having a 30 cm² active area. The devices were applied to the skin on the upper outer arm of human subjects. Blood samples were drawn from test subjects at regular intervals and tested for pergolide levels. Pergolide blood serum levels were determined by subjecting blood samples to liquid chromatography using mass spectroscopic detection according to testing protocol #AN47849-101-PPD.

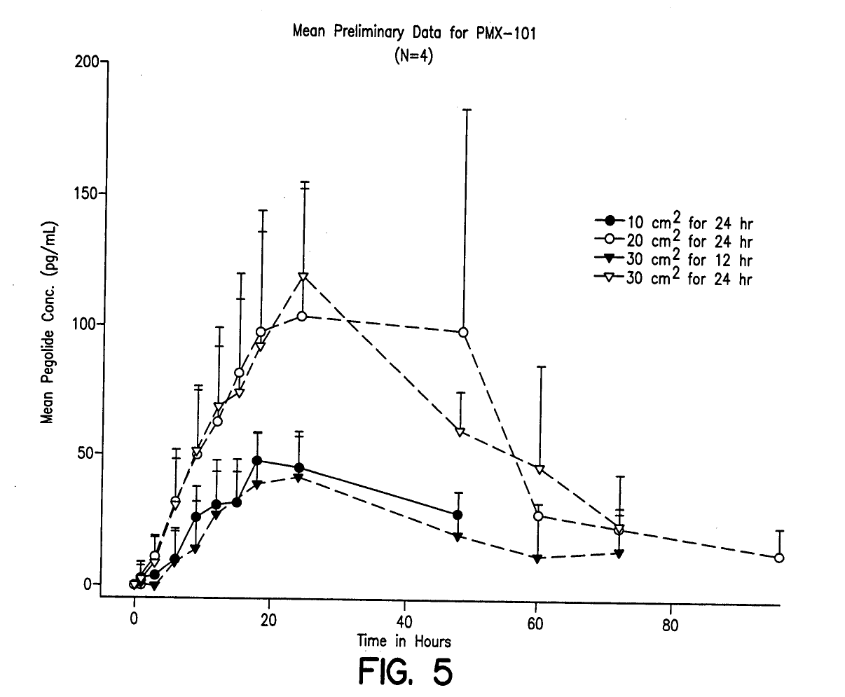

Blood serum levels obtained from samples from the 4 subjects wearing devices having the same pad size were averaged. The averaged data, along with the average of the times at which the samples were obtained are set forth in Table 2. Additional averaged data from this study is presented graphically in FIG. 5.

TABLE 2

| | | PERGOLIDE BLOOD |
|---|---|---|
| | | SERUM LEVEL |
| | | [mean of 4 subjects/ |
| EX. NO. | SURFACE AREA OF PAD* | picograms/ml] |
| 10 | 10 cm² | 48.9 |
| | | 19.5 hours after application |
| 11 | 20 cm² | 133.7 |
| | | 28.5 hours after application |
| 12 | 30 cm² | 42.2 |
| | | 22.5 hours after application |
| 13 | 30 cm² | 119.4 |
| | | 24 hours after application |

*Surface area determined by measurement of the area of the transdermal device containing the liquid composition of pergolide mesylate

For the purposes of comparison, there is set forth in Table 3 below representative examples of blood serum levels observed after oral dosing of human subjects with pergolide mesylate.

TABLE 3

| | Pergolide Blood |
|---|---|
| Oral Dose | Serum Level |
| [milligrams] | [picograms/ml] |
| 0.25 | 47.75 |
| 0.50 | 100.08 |
| 1.00 | 199.33 |
| 2.00 | 398.66 |
| 3.00 | 597.99 |

A comparison of the results reported in Tables 2 and 3 above reveals that transdermal devices of the present invention deliver therapeutic levels of pergolide mesylate. Additionally, the transdermal devices applied to human subjects did not produce skin irritation at the site of application.

The next group of examples are comparative in nature.

Transdermal devices having a matrix comprising pergolide base and enhancers were fabricated by blending pergolide free base and enhancers into aliquots of acrylate-based pressure sensitive adhesive (PSA) and casting the composition into a film in which the pergolide free base was present as a solid solution. Cast film matricies were prepared using National starch adhesives 87-2074 (Matrix A), 87-2620 (Matrix B), and 87-2696 (Matrix C) and Monsanto Adhesive 2873 Gelvea multipolymer acrylic resin (Matrix D). Matricies A, B, and D had a composition of 2 wt. % pergolide free base and 98.0 wt. % of the respective PSA used. The composition of Matrix C was 5 wt. % pergolide free base and 95 wt. % of the PSA used. The cast films were adhesive to skin as cast.

These cast films were cut into 1 cm² units. The pergolide delivery flux available from these cast film matricies was determined by adhering a laminate containing a 1 cm² unit to a sample of human skin mounted on a diffusion cell according to the in vitro testing procedure described above.

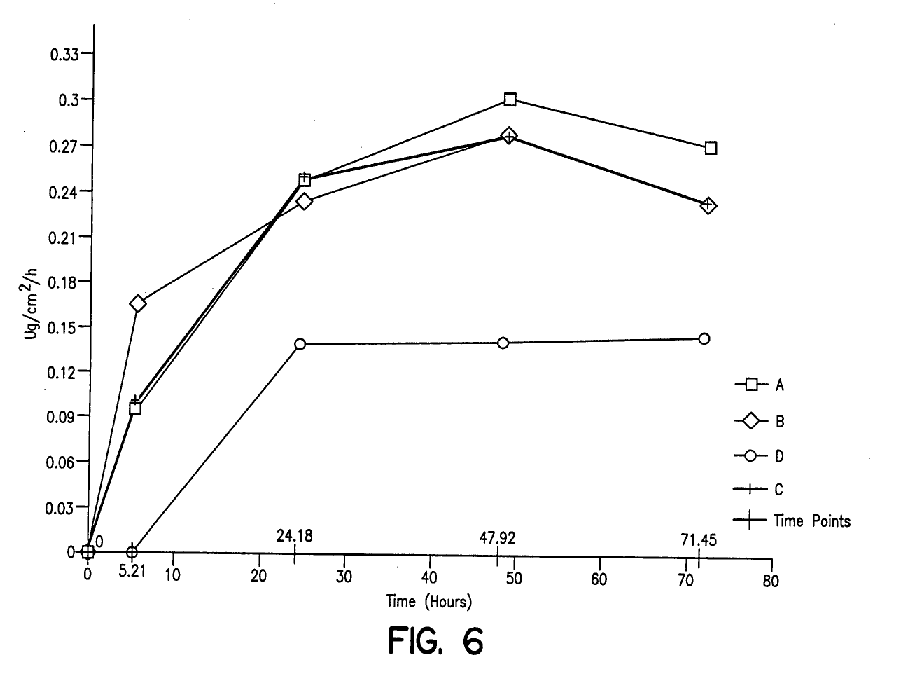

The results of these determinations are shown graphically in FIG. 6 as traces A-D for matricies A-D respectively. The results from these examples when compared with those examples reported above show that even at 4× the wt % pergolide loading, pergolide delivery flux from solid solutions is too low to be effective in yielding therapeutic blood serum levels of pergolide for the treatment of Parkinson's disease.

The next group of examples are illustrative of pergolide-containing compositions which can be used in the practice of the present invention. The compositions are identified in Table 4 below. The compositions were prepared by placing a weighted amount of the indicated solvent or solvents into a suitable mixing vessel and adding solid pergolide mesylate in the form of anhydrous powder and the additives identified in Table 4 into the solvent with stirring at 25° C. until the solid constituents dissolved. The various compositions prepared are reported in Table 4 as wt. % of the listed constituents.

TABLE 4

| | Pergolide Mesylate, | ENHANCER, | SOLVENT, Wt. % | | |
|---|---|---|---|---|---|
| Comp. No. | WT. % | WT. % | WATER | SOLVENT A | SOLVENT B |
| A | 2.5 | HPC¹ [4.0] | 79.0 | ethanol (14.5) | — |
| B | 1.2 | HPC¹ [5.0] | 93.8 | — | — |
| C | 1.2 | — | 98.8 | — | — |
| D | 1.7 | — | — | ethanol (16.6) | phospholipon |
| | | | | | (81.7) |
| E | 0.6 | — | — | ethanol (37.4) | GMO² (62.0) |
| F | 2.2 | Gelatin | 90.8 | — | — |
| | | (7.0) | | | |
| G | 0.9 | — | 92.1 | PVA³ (7.0) | — |
| H | 0.8 | Gelatin/7.9 | 59.0 | DMSO⁴ (30.8) | — |
| | | HPC¹/5.0 | | | |
| I | 1.0 | BCD⁵ (2.0) | 97.0 | — | — |

¹HPC = hydroxypropyl cellulose
²GMO = glycerol monooleate
³PVA = poly(vinyl alcohol)
⁴DMSO = dimethyl sulfoxide
⁵BCD = β cyclodextrin

An aliquot of each liquid pergolide composition identified in Table 4 above was applied directly to a section of human skin mounted on a diffusion cell. The delivery flux available from the composition was determined according to the in vitro method described above. The results of these determinations are summarized in Table 5, which also references figures containing additional data.

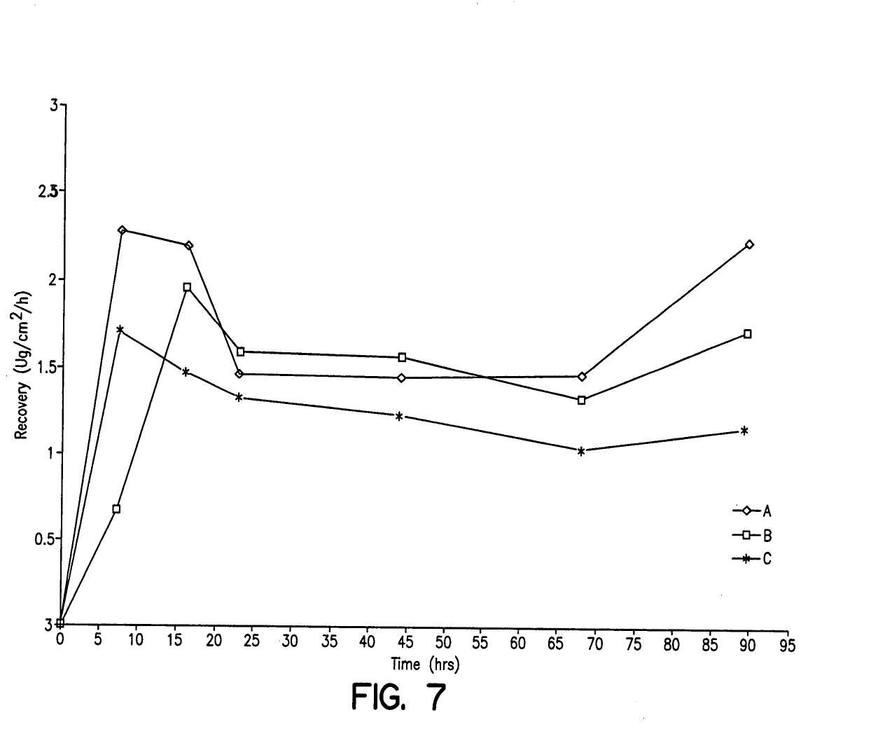

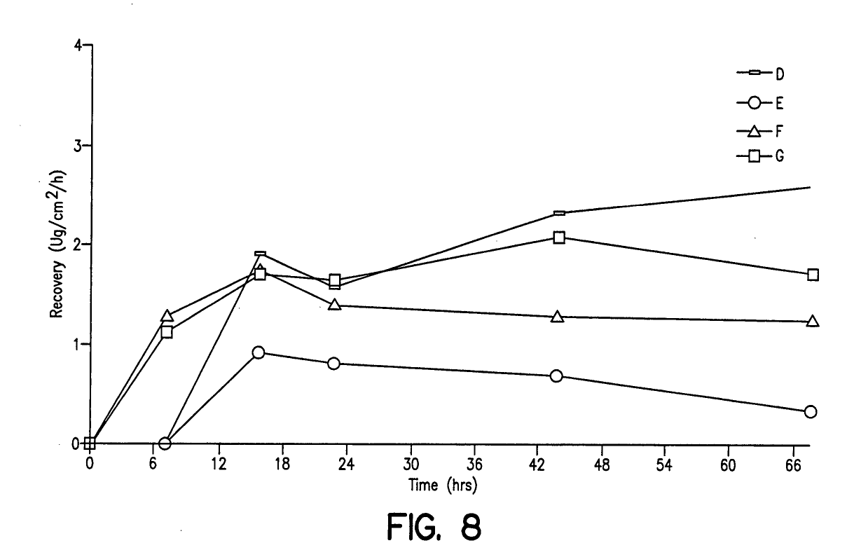

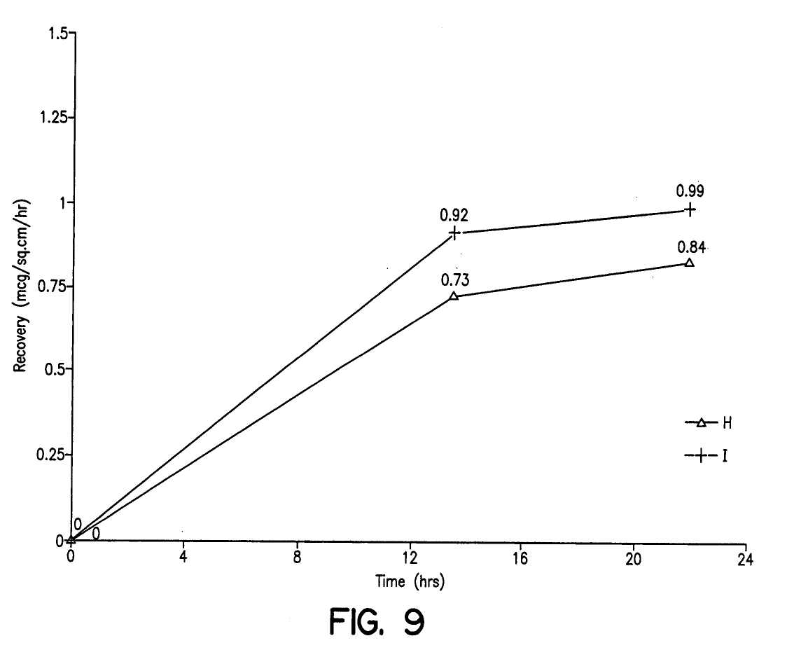

TABLE 5

| | Pergolide Delivery Flux | | |
|---|---|---|---|
| | (μg cm⁻² hr⁻¹) | | |
| Comp. No. | Initial/hours | Peak/ | Steady State/ |
| FIG. # | post application | hours post application | hours post application |
| A | 2.25/7 | 2.25/7 and again at | 1.45/24-60 |
| FIG. 7 - A | | 85 | |
| B | 1.9/15 | 1.9/15 | 1.6/24-45 |
| FIG. 7 - B | | | |
| C | 1.7/6 | 1.7/6 | 1.2/fluxuates 40-85 hours |
| FIG. 7 - C | | | post application |
| D | 1.9/14 | increasing steadily | no steady state |
| FIG. 8 - D | | between 20 and 60 hours | |
| | | from 1.7-2.5 | |
| E | 0.95/15 | 0.95/15 | 0.9-0.8/22-45 |
| FIG. 8 - E | | | |
| F | 1.2/7 | 1.6/16 | 1.4-1.3/22-66 |
| FIG. 8 - F | | | |
| G | 1.1/7 | 2.1/44 | 1.75-2.1/24-44 steady |
| FIG. 8 - G | | | rise to peak then decline |
| H | no peak | increasing throughout test | 0.75-0.8/steady rise |
| FIG. 9 - H | | | 12-21 hours (test duration) |
| I | no peak | increasing throughout test | 0.9-1.0/steady rise |
| FIG. 9 - I | | | 12-21 hours (test duration) |

In a separate experiment, composition D (Table 5), which contains no water, was applied to a 1 cm² pad of 1603 non-woven medical absorbent (3M) and fixed to a skin sample mounted on a diffusion cell. An in vitro determination of the delivery flux from this patch showed a peak flux of 0.05 μg/cm² hr of pergolide after 21 hours. The compositions of Table 5 can be used in a transdermal delivery device to treat patients.

1. A transdermal delivery device comprising a multi-phase matrix which includes a solid liquid-retaining member and associated therewith pergolide in liquid form.

2. The delivery device of claim 1 wherein said member comprises an absorbent material.

3. The delivery device of claim 1 including a composition comprising a liquid solution containing a liquid solvent and dissolved therein a solid form of pergolide.

4. The delivery device of claim 3 wherein said composition comprises an emulsion which has a continuous liquid phase in which droplets of said liquid solution are dispersed.

5. The delivery device of claim 1 wherein said matrix includes also an enhancer in liquid form.

6. The delivery device of claim 1 wherein said member comprises one or more of a natural fiber gauze, a synthetic fiber gauze, a non-woven medical absorbent, a natural sponge, and a synthetic sponge.

7. The delivery device of claim 1 wherein said matrix includes a composition comprising a liquid solvent and pergolide mesylate dissolved in said solvent.

8. The delivery device of claim 1 wherein said matrix includes a composition comprising one or more liquid solvents and dissolved therein said pergolide and an enhancer.

9. The delivery device of claim 6 wherein said absorbent member is a medical absorbent having a non-woven rayon web coated with a film of porous polyethylene.

10. The delivery device of claim 3 wherein the liquid solvent includes water.

11. The delivery device of claim 10 wherein the liquid solvent includes a cosolvent.

12. The delivery device of claim 11 wherein the cosolvent is an alcohol.

13. The delivery device of claim 3 wherein the solution includes also dispersed solid particles of pergolide.

14. The delivery device of claim 1 further comprising: (A) a barrier layer affixed to one face of said matrix and having an exposed face; (B) a mounting layer affixed to the exposed face of said barrier layer; and (C) a removable layer affixed to the other face of said matrix.

15. A method for the transdermal delivery of pergolide comprising delivering to a body membrane pergolide in liquid form and from a liquid pergolide-carrying member which is in direct contact with the body membrane.

16. The delivery device of claim 3 wherein said solution comprises:
(A) about 0.1 to about 1 wt. % pergolide mesylate;
(B) about 0.4 to about 2 wt. % b-cyclodextrine;
(C) about 3 to about 6 wt. % hydroxypropylcellulose; and
(D) about 90 to about 96 wt. % water.

17. The delivery device of claim 16 wherein said solution further comprises up to about 0.4 wt. % of an antioxidant.

18. The delivery device of claim 17 wherein the antioxidant is selected from the group consisting of ascorbic acid and citric acid or a mixture thereof.

19. The delivery device of claim 16 wherein said solution further comprises up to about 4 wt. % of a preservative.

20. The delivery device of claim 19 wherein the preservative is selected from the group consisting of benzyl alcohol and lactic acid or a mixture thereof.

21. The delivery device of claim 16 wherein said solution further comprises up to about 5 wt. % of ethanol.

22. The delivery device of claim 21 wherein said solution further comprises up to about 2 wt. % of an antioxidant selected from the group consisting of ascorbic acid and citric acid or a mixture thereof.
